(19) United States
(12) Patent Application Publication
(10) Pub. No.: US 2023/0313191 A1
(43) Pub. Date: Oct. 5, 2023

(54) RNA COMPRISING SECRETOMES AND METHODS OF THEIR USE

(71) Applicant: Direct Biologics LLC, Austin, TX (US)

(72) Inventors: Kevin Hicok, San Diego, CA (US); Timothy Alexander Moseley, Fallbrook, CA (US); Emil J. Schmidt, IV, St. Louis, MO (US)

(21) Appl. No.: 18/192,593

(22) Filed: Mar. 29, 2023

Related U.S. Application Data
(60) Provisional application No. 63/324,910, filed on Mar. 29, 2022.

(51) Int. Cl. C12N 15/113; A61P 37/02

(52) U.S. Cl. CPC C12N 15/113; A61P 37/02; C12N 2310/11; C12N 2310/32

(57) ABSTRACT

Disclosed are methods and compositions related to unique combinations of 2 or more RNA sequences including but not limited to microRNA, mRNA, long non-coding RNA, y-RNA, pi-RNA and other non-coding RNA packaged within naturally produced mesenchymal stem/stromal cell extracellular vesicles (EVs), synthetic EVs (aka liposomes or lipid nanoparticles), or in combination thereof that are capable of modifying immune function in a subject.

This PCT application claims priority to, and the benefit of, U.S. Provisional Patent Application No. 63/324,910, filed Mar. 29, 2022, which is incorporated by reference herein in its entirety.

### I. REFERENCE TO SEQUENCE LISTING

The sequence listing submitted on Mar. 29, 2023, as an .XML file entitled “11071-013US1.XML” created on Mar. 29, 2023, and having a file size of 2914 bytes is hereby incorporated by reference pursuant to 37 C.F.R. § 1.52(e)(5)

### II. BACKGROUND

Bone marrow mesenchymal stem/stromal cells (MSCs) modify the body's immune response primarily by secreting a combination of soluble factors, extracellular matrices, and extracellular vesicles (EVs). The EVs contain proteins and RNA molecules that regulate both proinflammatory and anti-inflammatory signaling pathways of immune cells (neutrophils, monocytes, macrophages, dendritic cells, B-cells, T-cells, natural killer (NK) cells, etc. Bacterial and viral pathogens have evolved strategies to manipulate the natural immune response to their advantage. For example, bacteria and viruses are able to induce interleukin 10 (IL-10), a primarily anti-inflammatory cytokine resulting in immune suppression, that enables the pathogen to increase their infection efficiency and replication rate. In other instances, a patient may have a genetically linked immune response deficit that may reduce the efficacy of the secreted factors and extracellular vesicles in the naturally derived therapeutic. These deficiencies may represent defects in the immunoregulatory signaling molecules or their receptors. What are needed are new compositions that can counteract microbial immune evasion techniques and immune response deficits.

### III. SUMMARY

Disclosed are methods and compositions related to unique combinations of 2 or more RNA sequences including but not limited to microRNA, mRNA, long non-coding RNA, y-RNA, pi-RNA and other non-coding RNA packaged within naturally produced mesenchymal stem/stromal cell extracellular vesicles (EVs), synthetic EVs (aka liposomes or lipid nanoparticles), or in combination thereof that are capable of modifying immune function in a subject.

In one aspect, disclosed herein are compositions comprising one or more secretomes (such as, for example, liposomes, lipid nanoparticles, or a mesenchymal stem cell (MSC) derived exosome or extracellular vesicle, or any combination thereof) and two or more ribonucleic nucleic acids (RNAs) (such as, for example, microRNA (miRNA), messenger RNA (mRNA), long non-coding RNA (lncRNA), y-RNA, and/or pi-RNA).

Also disclosed herein are compositions of any preceding aspect, wherein the two or more RNA comprises at least one RNA selected from the group consisting of RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and U7:RF00066.146.

In one aspect, disclosed herein are compositions of any preceding aspect, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions of any preceding aspect, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p.

Also disclosed herein are compositions of any preceding aspect, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS.

In one aspect, disclosed herein are compositions of any preceding aspect, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy21.

Also disclosed herein are compositions of any preceding aspect, wherein the two or more RNA comprises at least one lncRNA selected from the group consisting of LOC100129335-5, ZNF238, CADM2-7, SALL1-6, and VWA5A.

In one aspect, disclosed herein are compositions of any preceding aspect, wherein the two or more RNA comprises at least one antisense lncRNA selected from the group consisting of ANKRD5-1, ZNF236-1, and ACP1.

Also disclosed herein are compositions of any preceding aspect, wherein the two or more RNA comprises at least one y-RNA selected from the group consisting of RF00019.441, RF00019.451, RF00019.621, RF00019.646, and RF00019.413.

In one aspect, disclosed herein are compositions of any preceding aspect, wherein the two or more RNA comprises at least one pi-RNA selected from the group consisting of piR-33044, piR-33043, and piR-31703.

Also disclosed herein are compositions of any preceding aspect, wherein the two or more RNA comprises at least one antisense RNA selected from the group consisting of EPHA1-AS1, FLJ45974, ANKRD30BP2, LINC00340, GRIK1-AS1, LOC440300, CSPG4P8, ADARB2-AS1, MLLT4-AS1, and HNRNPU-AS1.

In one aspect, disclosed herein are compositions of any preceding aspect, wherein the RNA comprises a modification to reduce degradation (such as, for example, the addition of 2′-O-Methyl, 2′-Fluoro or 2′-Hydro groups).

Also disclosed herein are method of modulating an immune response (including, but not limited to increasing or decreasing inflammation) to an inflammatory injury in a subject comprising administering to the subject any of the compositions of any preceding aspect. For example, disclosed herein are methods of modulating an immune response, wherein the modulation increases inflammation and wherein the two or more RNA of the composition comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also for example, disclosed herein are methods of modulating an immune response, wherein the modulation decreases inflammation and wherein the two or more RNA of the composition comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

In one aspect, disclosed herein are methods of treating, inhibiting, decreasing, reducing, ameliorating and/or preventing an autoimmune disease, autoinflammatory disease, inflammatory injury from a physical insult, inflammatory injury resulting from a microbial infection, or inflammatory injury resulting from a cancer in a subject comprising administering to the subject the composition of any preceding aspect.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effect of an MSC extracellular vesicle preparation EXOFLO® and synthetic miRNAs shown by RNA sequencing and quantitative PCR to be present in EXOFLO® on IL-1β concentration (n=3 wells of cells per condition).

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular composition comprising one or more secretomes and two or more ribonucleic nucleic acids (RNAs) is disclosed and discussed and a number of modifications that can be made to a number of molecules including the composition comprising one or more secretomes and two or more ribonucleic nucleic acids (RNAs) are discussed, specifically contemplated is each and every combination and permutation of composition comprising one or more secretomes and two or more ribonucleic nucleic acids (RNAs) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Bone marrow mesenchymal stem/stromal cells (MSCs) modify the body's immune response primarily by secreting a combination of soluble factors, extracellular matrices, and extracellular vesicles (EVs). The EVs contain proteins and RNA molecules that regulate both proinflammatory and anti-inflammatory signaling pathways of immune cells (neutrophils, monocytes, macrophages, dendritic cells, B-cells, T-cells, natural killer (NK) cells, etc. Bacterial and viral pathogens have evolved strategies to manipulate the natural immune response to their advantage. For example, bacteria and viruses are able to induce interleukin 10 (IL-10), a primarily anti-inflammatory cytokine resulting in immune suppression, that enables the pathogen to increase their infection efficiency and replication rate. In this example, we propose that the supplementation of the naturally derived, non-engineered EV therapeutic at temporally appropriate times along with engineered synthetic miRNA, RNAi or proteins targeted against molecules like IL-10, that are enabling the sustained infection, may generate a more favorable resolution to the hyper- or hypo-immune disease state.

In other instances, a patient may have a genetically linked immune response deficit that may reduce the efficacy of the secreted factors and extracellular vesicles in the naturally derived therapeutic. These deficiencies may represent defects in the immunoregulatory signaling molecules or their receptors. Using IL-10 again as an example, individuals with predisposition toward autoimmunity upon microbial challenge generate an auto-immune response when IL-10 is suppressed (Gazzinelli et al). To counteract that, we propose that additional supplementation of an appropriately engineered IL-10 regulatory RNA or protein would drive an enhanced effect of the immune response than that achieved by the naturally derived therapeutic alone. This therapeutic strategy could at the same time allow for the adequate compensation of the genetically dysregulated immune function within the autoimmune prone patient.

In the above scenarios, the proposed use and/or delivery of therapeutic EV/secreted factors along with other engineered molecules to the patient allows for precise tailored immunotherapy and that is otherwise not achievable through delivery of the individual elements alone. Therefore, through the addition of synthetic biomolecules to the naturally derived EV/secreted factor-based therapy, a customized, tuned effect that increases efficacy across a broader patient population can be achieved.

In one aspect, disclosed herein are compositions comprising one or more secretomes (such as, for example, liposomes, lipid nanoparticles, or a mesenchymal stem cell (MSC) derived exosome or extracellular vesicle, or any combination thereof) and two or more ribonucleic nucleic acids (RNAs) (such as, for example, microRNA (miRNA), messenger RNA (mRNA), long non-coding RNA (lncRNA), y-RNA, and/or pi-RNA).

As noted throughout, the compositions disclosed herein can utilize liposomes, lipid nanoparticles, as well as, exosomes and/or extracellular vesicles derived from mesenchymal stem cells (MSCs), or any combination thereof. While existing autogenous and allogeneic MSCs contained within bone marrow concentrate or adipose-derived stromal vascular fraction (SVF) or various post-natal products from umbilical cord, placenta or amnion, expanded MSC cultures are currently being used to treat wounds, orthopedic pathology, and spine pathology; the existing treatments do not contain large amounts of MSC secretomes (including, but not limited to growth factors, cytokines, chemokines, exosomes, extracellular vesicles, and/or extracts).

MSC are multipotent cells that have the ability to differentiate into a multitude of cell types including myocytes, chondrocytes, adipocytes, and osteoblasts. Typically, these cells can be found in the placenta, umbilical cord blood, adipose tissue, bone marrow, or amniotic fluid, including perivascular tissue. As used herein, “MSC” refers to non-terminally differentiated cells including but not limited to multipotential stem cell, multipotential stromal cell, stromal vascular cells, pericytes, perivascular cells, stromal cells, pluripotent cells, multipotent cells, adipose-derived fibroblast-like cells, adipose-derived stromal vascular fraction, adipose-derived MSC, bone marrow-derived fibroblast-like cells, bone marrow-derived stromal vascular fraction, bone marrow-derived MSC, tissue-derived fibroblast-like cells, adult stem cells, adult stromal cells, keratinocytes, and/or melanocytes.

It has been long recognized that MSC, in addition to their differentiation potential, have the immunomodulatory abilities resulting in the expression of many different cytokines and growth factors. As used herein, a “MSC preparation” or “MSC secretome composition” refers to a composition comprising MSC growth factors, MSC exosomes, extracellular vesicles, or acellular extracts of MSCs or MSC lysates obtained from human MSCs, fibroblast-like cells, and non-human animal MSCs including, but not limited to MSCs from horses, cows, pigs, sheep, non-human primates, dogs, cats, rabbits, rats, and mice. In embodiments, the MSCs may be derived from the patient to which the composition will be applied (autologous) or derived from another individual (allogeneic). The MSCs may be culture expanded to collect the conditioned media or to increase the quantity of cells for the lysate or used freshly prior to incorporation into the composition of the present disclosure.

The MSC secretome compositions (including, but not limited to MSC growth factor, MSC exosome, MSC extracts and/or extracellular vesicle comprising compositions) may comprise about 0.00001 to about 20 wt. %, such as from about 0.01 to about 10 wt. %, of a mesenchymal stem cell (MSC) extract, MSC exosome, or MSC growth factor preparation. The MSC preparation may comprise either MSC conditioned media or MSC lysate from cell culture expanded MSCs. In some embodiments, the composition may further comprise from about 0.01 to about 10 wt. % of a cell-free medium conditioned by growth of MSCs or MSC lineage cells, wherein the cells are cultured under normal hyperoxyic culturing conditions or under artificial wound healing conditions.

As disclosed herein the MSCs used to produce the disclosed MSC additives (including growth factor secretome composition either frozen or powdered additives) can be selectively stimulated to produce MSC growth factors, secretomes, cytokines, chemokines, mesenchymal stem cell proteins, peptides, glycosaminoglycans, extracellular matrix (ECM), proteoglycans, secretomes, and exosomes. As used herein, MSC growth factors include but are not limited to prostaglandin E2 (PGE2), transforming growth factor β1 (TGF-β1), hepatocyte growth factor (HGF), stromal cell derived factor-1 (SDF-1), nitric oxide, indoleamine 2,3-dioxygenase, interleukin-4 (IL-4), IL-6, interleukin-10 (IL-10), IL-1 receptor antagonist and soluble TNF-α receptor, insulin-like growth factors, fibroblast growth factors (FGF) 1-23 (especially, FGF1 and FGF2), bone morphogenetic proteins (BMPs) 1-15, epidermal growth factor (EGF), transforming growth factor-α (TGF-α) macrophage-stimulating protein (MSP), platelet derived growth factor (PLGF), vascular endothelial growth factor (VEGF), macrophage colony stimulating factor (M-CSF), insulin, granulocyte colony stimulating factor (G-CSF), granulocyte macrophage colony stimulating factor (GM-CSF), as well as hormones including estrogen, and thyroid hormones.

In one aspect, the MSC preparation (such as, for example, a MSC secretome composition) comprises MSC growth factors, MSC exosomes, and/or cellular extracts of MSCs or MSC lysates obtained from MSCs cultured under standard hyperoxyic culturing conditions (for example, 21% oxygen) or MSCs cultured under artificial wound healing conditions (such as, for example, 0.1% to about 5% oxygen in the presence of inflammatory cytokines, angiogenic factors, and reduced glucose).

As disclosed herein artificial wound healing conditions simulate growth conditions in real wounds where there is a reduction in nutrient supply and reduction of waste removal that is usually caused by a disruption in local blood circulation. This creates a harsh environment for cells until new blood vessels are created and blood circulation is restored. Accordingly, artificial wound healing conditions used to culture MSCs can include one or more of the following growth conditions reduction in glucose availability, reduction in oxygen tension, reduction in pH, and increased temperature.

In one aspect, the glucose availability can be reduced relative to normal control. Modified culture media to reduce glucose, but not damage the cells can be between 0 and 50% reduction in glucose, more preferably between about 5% and 40% reduction in glucose. For example, MSC artificial wound healing culture conditions can comprise glucose reduction of about 1, 2, 3, 4, 5, 6, 7, 8 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% such as a glucose reduction from about 5% to about 15%, from about 10% to about 20%, from about 15% to about 25%, from about 20% to about 30%, or from about 25% to about 35%.

In one aspect, oxygen tension can be reduced to oxygen levels to hypoxic conditions. Normal atmospheric oxygen is approximately 21% and any reduction is considered hypoxic. Thus, in one aspect, MSCs can be cultured at between 0.0% and 20.9% oxygen, from about 0.1% to about 0.5% oxygen, from about 0.1% to about 2.0%, from about 0.1% to about 5.0% oxygen, from about 0.5% to 5.0%, from about 1.0% to about 10% oxygen, about 5.0% to about 10.0% oxygen; and from about 10.0% to about 15.0% under artificial wound healing conditions. Preferably during MSC would healing culture conditions oxygen tension is between about 0.5% and 20.5% oxygen, such as, for example, 0, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.7, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, or 20.5% oxygen.

The pH can also be reduced under artificial wound healing conditions. Physiologic pH is maintained very tightly and is usually very close to a neutral pH=7.2±0.2 (7.0-7.4). However, in a wound the acidic environment can have a pH=6.2±0.2 (i.e., a pH from 6.0 to about 6.4). Thus, under artificial wound healing culture conditions, pH can be from about 6.0 to about 7.4, for example, from 6.0 to about 6.4, from about 6.2 to about 6.4, from about 6.2 to about 6.6, from about 6.4 to about 6.6, from about 6.4 to about 6.8, or from about 6.6 to about 7.0, such as 6.0, 6.1, 6.2, 6.3, 6.4, 6.5, 6.6, 6.7, 6.8, 6.9, 7.0, 7.1, 7.2, 7.3 or 7.4.

Under artificial wound healing culture conditions, the temperature of the culture environment may be raised to simulate temperature increases at the site of a wound. Physiologic homeostasis temperature is maintained at 37° C. (98.6° F.). A slight increase or decrease can cause significant changes to cellular metabolism. By increasing the temperature above 37° C. to any temperature up to about 40° C. (104° F.) can create an “feverous” environment. Thus, in on aspect, the artificial wound healing culture conditions for the MSCs can comprise from about 35° C. to about 39° C., from about 35° C. to about 36° C., from about 36° C. to about 37° C., from about 37° C. to about 38° C., from about 38° C. to about 39° C., from about 39° C. to about 40° C. In one aspect, the temperature of the artificial wound healing culture can be 35.0, 35.1, 35.2, 35.3, 36.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, or 40.0° C.

The combined reduced nutrient and metabolite environment (artificial wound healing) can trigger the cultured cells to produce wound healing and anti-inflammatory ECM proteins and growth factors and extracellular vesicles that are there to direct tissue healing, which can be in the form of new ECM proteins, such as collagen and glycosaminoglycans (GAGs) as well as growth factors and cytokines. Thus, it is understood and herein contemplated that by adjusting the cell growth conditions, such as cell confluency, culture media supplements, nutritional supplements, oxygen levels, length of culture in those conditions, cell passage number or combinations of those, and the like, MSCs can be stimulated to selectively secrete the desired anti-inflammatory proteins, peptides, cytokines, chemokines, glycosaminoglycans, extracellular matrix (ECM), proteoglycans, exosomes and secretomes.

In one aspect, it is understood and herein contemplated that the growth conditions such as temperature, oxygen tension, pH, glucose saturation, confluency, and growth surface can affect the gene expression and protein production of cells growing in culture and thereby can result in different growth factors and cytokines being produced. For example, growth surface stiffness (Young's Modulous) affects the gene expression and protein production of the cells growing on it. Adipose cells and cartilage cells are usually maintained on a softer and more elastic growth surface (˜10 kPa-12 kPa), while bone cells are better grown on a stiff surface (˜10⁶-12⁶ kPa). By adjusting the surface stiffness, it is possible to influence the secretomes of the cells and their communication signals (growth factors, exosomes, cytokines and chemokines).

In one aspect, the secretome compositions (including, but not limited to liposomes, lipid nanoparticles, MSC growth factor, MSC exosome, MSC extracts and/or extracellular vesicle comprising compositions) can further comprise a protective coating (such as, for example, a cryoprotectant oligosaccharide and a protein solution) to reduce degradation of the growth factors. It is understood and herein contemplated that the protective coating can be engineered as a polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc. In one aspect, the gel matrix can comprise copolymers, block copolymers, diblock copolymers, and/or triblock copolymers.

In one aspect, the protective coating can comprise a biocompatible polymer. In one aspect, biocompatible polymer can be crosslinked. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

In some embodiments the protective coating comprises carbohydrate construction of monosaccharides as well as carbohydrate polymers such as disaccharides or polysaccharides including but not limited to non-reducing poly or disaccharides as well as any combination thereof. Examples of carbohydrates that can be used in the protective coating comprise Glucose, Aldoses (D-Allose, D-Altrose, D-Mannose, etc.), Glucopyranose, Pentahydroxyhexanal, a-D-Glucopyranosyl-D-glucose, α-D-Glucopyranosyl-dihydrate, Polymer of β-D-Glycopyranosyl units, β-D-Fructofuranosyl α-D-glucopyranoside (anhydrous/dihydrate), β-D-Galactopyranosyl-D-glucose, α-D-Glucopyranosyl-α-D-glucopyranoside (anhydrous/dihydrate), Galactose, Pentoses (Ribose, xylose, lyxose), Dextrose, Dodecacarbon monodecahydrate, Fructose, Sucrose, Lactose, Maltose, Trehalose, Agarose, D-galactosyl-β-(1-4)-anhydro-L-galactosyl, Cellulose, Polymer of β-D-Glycopyranosyl units, and Starch, as well as, Polyhydric alcohols, Polyalcohols, Alditols, Erythritol, Glycitols, Glycerol, Xylitol, and Sorbitol.

In some embodiments the protective coating contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

Examples of diblock copolymers that can be used in the protective coatings disclosed herein comprise a polymer such as, example, polyethylene glycol (PEG), polyvinyl acetate, polyvinyl alcohol (PVA), polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA).

In one aspect, the protective coating contains (i.e., the encapsulated, the encapsulated compositions can further comprise lecithin or hydrolyzed lecithin as a carrier or as encapsulation material. As used herein, lecithin and/or hydrolyzed lecithin coatings include coatings comprising phosphatidyl choline, phosphatidyl inositol, phosphatidyl ethanolamine, phosphatidylserine, and phosphatidic acid. Sources of the lecithin can be plant or animal sources.

In one aspect, any of the polymers, monosaccharides, disaccharides, or polysaccharides used to form the protective coating formed by placing the MSC additive in a encapsulating solution can be at an appropriate concentration for form the protective coating. For example, polymers, monosaccharides, disaccharides, or polysaccharides can be at any concentration between 0.01 mM and 10.0M concentration, for example, from about 0.01M to about 0.1M, from about 0.1 mM to about 1.0M, from about 1.0M to about 10.0M. Exemplary concentrations include 0.01, 0.02, 0.03, 0.04, 0.05, 0.06, 0.07, 0.08, 0.09, 0.1, 0.2, 0.3, 0.4, 0.4, 0.6, 0.7, 0.8, 0.9, 1, 2, 3, 4, 5, 6, 7, 8, 9, 10, 20, 30, 40, 50, 60, 70, 80, 90, 100, 110, 120, 130, 140, 150, 160, 170, 180, 190, 200, 225, 250, 275, 300, 325, 350, 375, 400, 450, 500, 600, 700, 800, 900 mM, 1, 1.1, 1.2, 1.3, 1.4, 1.5, 1.6, 1.7, 1.8, 1.9, 2, 3, 4, 5, 6, 7, 8, 9, 10M.

The disclosed compositions achieve much of their inflammation modulation ability from the inclusion of two or more ribonucleic acid molecules (such as, for example, microRNA (miRNA), messenger RNA (mRNA), long non-coding RNA (lncRNA), y-RNA, and/or pi-RNA). Examples of the RNAs that can be used in the disclosed compositions include, but are not limited to hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146. In some aspects, at least one of the two or more RNAs can comprise an antisense RNA. Examples of antisense RNA that can be used in the disclosed compositions include, but are not limited to UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1. Accordingly, in one aspect, disclosed herein are compositions comprising one or more secretomes and two or more RNAs, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p.

Also disclosed herein are compositions comprising one or more secretomes and two or more RNAs, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS.

In one aspect, disclosed herein are compositions comprising one or more secretomes and two or more RNAs, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy2l.

Also disclosed herein are compositions comprising one or more secretomes and two or more RNAs, wherein the two or more RNA comprises at least one lncRNA selected from the group consisting of LOC100129335-5, ZNF238, CADM2-7, SALL1-6, and VWA5A.

In one aspect, disclosed herein are compositions of any preceding as comprising one or more secretomes and two or more RNAs pect, wherein the two or more RNA comprises at least one antisense lncRNA selected from the group consisting of ANKRD5-1, ZNF236-1, and ACP1.

Also disclosed herein are compositions comprising one or more secretomes and two or more RNAs, wherein the two or more RNA comprises at least one y-RNA selected from the group consisting of RF00019.441, RF00019.451, RF00019.621, RF00019.646, and RF00019.413.

In one aspect, disclosed herein are compositions comprising one or more secretomes and two or more RNAs, wherein the two or more RNA comprises at least one pi-RNA selected from the group consisting of piR-33044, piR-33043, and piR-31703.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

As noted throughout, in addition to MSC derived exosomes or extracellular vesicles, the disclosed RNAs can be comprised and delivered to a subject via lipids such as lipid nanoparticles or liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

In one aspect, the secretome compositions (including, but not limited to liposomes, lipid nanoparticles, MSC growth factor, MSC exosome, MSC extracts and/or extracellular vesicle comprising compositions) disclosed herein may further comprise any known ingredients typically found in the wound healing fields, such as oils, waxes or other standard fatty substances, or conventional gelling agents and/or thickeners; emulsifiers; moisturizing agents; emollients; sunscreens; hydrophilic or lipophilic active agents, such as ceramides; agents for combating free radicals; bactericides; sequestering agents; preservatives; basifying or acidifying agents; fragrances; surfactants; fillers; natural products or extracts of natural product, such as aloe or green tea extract; vitamins; or coloring materials. Other ingredients that may be combined with the powder may include an antioxidant, which can be selected from a variety of antioxidants. Suitable antioxidants include vitamins, such as Vitamin C (L-Ascorbate, Ascorbate-2 Phosphate magnesium salt, Ascorbyl Palmitate, Tetrahexyldecyl Ascorbate), Vitamin E (Tocotrienol), Vitamin A (retinol, retinal, retinoic acid, provitamin A carotenoids, such as beta-carotene), N-acetyl glucosamine, or other derivatives of glucosamine. Other ingredients may include at least one essential fatty acid, such as Ω-3, Ω-6, and Ω-9 polyunsaturated fatty acids, such as linoleic acid (LA), gamma-linoleic acid (GLA), alpha-linoleic acid (ALA), dihomo-y-linolenic acid (DGLA), arachidonic acid (ARA), and others. The fatty acids may be derived from various sources including evening primrose oil, black currant oil, borage oil, or GLA modified safflower seeds. Other ingredients may include a platelet rich fibrin matrix, at least one ingredient to support ECM production and production of hyaluronic acid, such as N-acetyl glucosamine or other derivatives of glucosamine, ultra-low molecular weight (ULMW) hyaluronic acid, chondroitin sulfate, or keratin sulfate.

The RNA loaded secretomes can be made by any natural or synthetic process known in the art. For example, naturally occurring RNA synthesis and vesicle loading processes within cells are used to generate RNA-loaded EVs. Alternatively, RNAs can be synthesized by de novo organic synthesis methods and loaded into artificial lipid nanoparticles. In another example RNA species in naturally-derived EVs are combined with artificial lipid nanoparticles loaded with synthetic RNA species at various combinatorial ratios of 1:1 to 1:10000 where various combinations are delivered as individual vesicles. In another example RNA species in naturally-derived EVs are combined with artificial lipid nanoparticles loaded with synthetic RNA species at various combinatorial ratios of 1:1 to 1:10000 where various combinations undergo 1:1 fusion to generate combined chimeric natural/synthesized EV particles containing combinations of natural and synthetic RNA sequences. In some configurations the synthetic RNA may be loaded into synthetic lipid nanoparticles along with co-factor proteins that enhance the RNA sequence function. It is understood and herein contemplated that naturally and synthetically generated secretomes can be used in the same composition. Accordingly, in one aspect, different combinations of naturally generated and synthetically generated EVs are dosed into recipient at different times to improve effects on genes expressed in either different cell types or when expressed at different times in the resolution progression of immune modulation.

1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and

RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

In some aspects, the RNA can be modified to reduce degradation. For example, the RNA can comprise the addition of 2′-O-Methyl, 2′-fluoro, or 2′-Hydro groups.

b) Sequences

The sequences for the human analogs of RNA molecules disclosed herein, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWASA, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR10P21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules (such as, for example, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy2l, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and HNRNPU-AS1) are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

4. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF MODULATING INFLAMMATION

The disclosed RNAs can target genes involved in immune modulation. For example, miR-92a-3p targets HAS2, NOX4, and/or ADAMTS-4; let-7i-5p targets HAS2, TLR4, NOX2, MMP1, and/or COX2; miR-16-5p targets HAS2, NOS1, and/or COX2; miR-93-5p targets HAS2, NOX2, and/or ADAMTS-4; miR-21-5p targets IL-1B, IL6, STAT3, PTEN, TNF, IRAK1, and/or CCL20; miR-214-3p targets CASP1; miR-29c-3p and miR-34a-5p target BDKRB2; mir-203a-3p targets, MMP-13, NOX2, and/or ADAMTS-4; miR-27b-3p targets, MMP-13, NOX2, and/or IL1B; miR-140-3p targets MMP-13, ADAMTS-4, and/or BCL2; miR-24-3p targets MMP-13, ADAMTS-4, and/or IL1B, miR-222-3p targets MMP-13 and/or MMP1; miR-25-3p targets MMP-13, NOX4, and/or ADAMTS-4; miR-10b-5p targets NOX2/ADAMTS-4; miR-221-3p targets MMP1 and/or IL1B; miR-17-5p targets the jak-stat pathway, PTEN, TNF, and/or CXCL8; miR-30a-5p targets inhibition of ESCC; and miR-30d-5p is a tumor suppressor in lung tumors.

Accordingly, also disclosed herein are method of modulating an immune response (including, but not limited to increasing or decreasing inflammation) to an inflammatory injury in a subject comprising administering to the subject any of the compositions disclosed herein including, but not limited to compositions comprising one or more secretomes (including, but not limited to liposomes, lipid nanoparticles, MSC growth factor, MSC exosome, MSC extracts and/or extracellular vesicle comprising compositions) and two or more RNA molecules comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

For example, disclosed herein are methods of modulating an immune response, wherein the modulation increases inflammation and wherein the two or more RNA of the composition comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2110, SMC4, RPL4, PSAP, ASIC4, AHCYL1, RD3L, SIGLEC1, SYCE2, and NANS. Also for example, disclosed herein are methods of modulating an immune response, wherein the modulation decreases inflammation and wherein the two or more RNA of the composition comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

### D. METHODS OF TREATING AUTOIMMUNE DISEASES

As noted above, the secretome and RNA compositions disclosed herein can modulate inflammation and arrest inflammation-driven destruction associated with diseases including autoimmune disease. Thus, the disclosed compositions can be used in the treatment of autoimmune disease or the inflammatory symptoms thereof. As used herein, “autoimmune disease” refers to a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells bearing an antigen for which they are specific. Examples of autoimmune diseases include, but are not limited to graft versus host disease, transplant rejection, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal pemphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)). In one aspect, disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR10P21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1. For example, disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p. Also disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one y-RNA selected from the group consisting of RF00019.441, RF00019.451, RF00019.621, RF00019.646, and RF00019.413. Also disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one lncRNA selected from the group consisting of LOC100129335-5, ZNF238, CADM2-7, SALL1-6, and VWA5A. Also disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one pi-RNA selected from the group consisting of piR-33044, piR-33043, and piR-31703. In one aspect, the RNA can be antisense RNA. Thus, also disclosed herein are methods of treating, inhibiting, decreasing, reducing, ameliorating and/or preventing autoimmune diseases or inflammatory symptoms associated with an autoimmune disease in a subject with an autoimmune disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR10P21, WBSCR271, NBPF141, TMEM132E1, GAGE131, GAGE2D1, and GAGE2D:copy2l, antisense lnRNA selected from the group consisting of ANKRD5-1, ZNF236-1, and ACP1, and/or antisense RNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy2l, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

### E. METHODS OF TREATING AUTOINFLAMMATORY DISEASES

The compositions comprising secretomes and RNA disclosed herein are not limited in treatment of inflammation resulting from adaptive immune responses but are also effective in arresting inflammation-driven destruction associated with the inborn errors of innate immune responses (i.e. Constitutive inflammation that underlies autoinflammatory diseases). As used herein “autoinflammatory diseases refer to disorders where the innate immune response attacks host cells. Examples of autoinflammatory disorders include, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF) and other cryopyrin-associated periodic syndromes (CAPS), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome and other Type 1 Interferonopathies, Acute Febrile Neutrophilic Dermatosis, X-linked familial hemophagocytic lymphohistiocytosis, Lyn kinase-associated Autoinflammatory Disease (LAID), and intestinal and skin inflammatory disorders caused by deletion mutation of the carboxy-terminal segment of the NF-κB essential modulator (NEMO). In one aspect, disclosed herein are methods of treating, inhibiting, decreasing, reducing, ameliorating and/or preventing autoinflammatory diseases or inflammatory symptoms associated with an autoinflammatory disease in a subject comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR10P21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1. For example, disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p.

Also disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one y-RNA selected from the group consisting of RF00019.441, RF00019.451, RF00019.621, RF00019.646, and RF00019.413. Also disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one lncRNA selected from the group consisting of LOC100129335-5, ZNF238, CADM2-7, SALL1-6, and VWA5A. Also disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one pi-RNA selected from the group consisting of piR-33044, piR-33043, and piR-31703. In one aspect, the RNA can be antisense RNA. Thus, also disclosed herein are methods of treating, inhibiting, decreasing, reducing, ameliorating and/or preventing autoinflammatory diseases or inflammatory symptoms associated with an autoinflammatory disease in a subject with an autoinflammatory disease comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy2l, antisense lnRNA selected from the group consisting of ANKRD5-1, ZNF236-1, and ACP1, and/or antisense RNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

### F. METHODS OF TREATING PHYSICAL INSULTS CAUSING PHYSICAL OR POST-TRAUMATIC INFLAMMATION

Many inflammatory conditions result from physical injuries mediated by inflammation (such as, for example abrasion, puncture, laceration, contusion, including brain trauma, blunt force trauma, ischemia, surgery, transplant, sunburn, chemical burn, high temperature burn, low temperature burn, radiation). As noted above, the secretome and RNA comprising compositions disclosed herein can modulate inflammation. Thus, the novel forms of immunotherapy disclosed herein can arrest inflammation-driven organ damage associated with these physical injuries that cause swelling, redness, elevated temperature, pain, and loss of organ function. Accordingly, in one aspect, disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) comprising administering to a subject with a physical injury a therapeutically effective amount of a composition comprising one or more secretomes and two or more RNAs comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203 a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR10P21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and HNRNPU-AS1.

methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) comprising administering to a subject with a physical injury a therapeutically effective amount of a composition comprising one or more secretomes and two or more RNAs, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p.

Also disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) or inflammatory symptoms associated with an inflammation caused by physical injury (such as, for example, abrasion, puncture, laceration, contusion, blunt force trauma, ischemia, surgery, transplant, sunburn, chemical burn, high temperature burn, low temperature burn) in a subject with an inflammation caused by physical injury (such as, for example, abrasion, puncture, laceration, contusion, blunt force trauma, ischemia, surgery, transplant, sunburn, chemical burn, high temperature burn, low temperature burn) comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) or inflammatory symptoms associated with an inflammation caused by physical injury (such as, for example, abrasion, puncture, laceration, contusion, blunt force trauma, ischemia, surgery, transplant, sunburn, chemical burn, high temperature burn, low temperature burn) in a subject with an inflammation caused by physical injury (such as, for example, abrasion, puncture, laceration, contusion, blunt force trauma, ischemia, surgery, transplant, sunburn, chemical burn, high temperature burn, low temperature burn) comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one y-RNA selected from the group consisting of RF00019.441, RF00019.451, RF00019.621, RF00019.646, and RF00019.413. Also disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) or inflammatory symptoms associated with an inflammation caused by physical injury (such as, for example, abrasion, puncture, laceration, contusion, blunt force trauma, ischemia, surgery, transplant, sunburn, chemical burn, high temperature burn, low temperature burn) in a subject with an inflammation caused by physical injury (such as, for example, abrasion, puncture, laceration, contusion, blunt force trauma, ischemia, surgery, transplant, sunburn, chemical burn, high temperature burn, low temperature burn) comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one lncRNA selected from the group consisting of LOC100129335-5, ZNF238, CADM2-7, SALL1-6, and VWA5A. Also disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) or inflammatory symptoms associated with an inflammation caused by physical injury (such as, for example, abrasion, puncture, laceration, contusion, blunt force trauma, ischemia, surgery, transplant, sunburn, chemical burn, high temperature burn, low temperature burn) in a subject with an inflammation caused by physical injury (such as, for example, abrasion, puncture, laceration, contusion, blunt force trauma, ischemia, surgery, transplant, sunburn, chemical burn, high temperature burn, low temperature burn) comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one pi-RNA selected from the group consisting of piR-33044, piR-33043, and piR-31703. In one aspect, the RNA can be antisense RNA. Thus, also disclosed herein are methods of treating, inhibiting, decreasing, reducing, ameliorating and/or preventing inflammation caused by physical injury (such as, for example, abrasion, puncture, laceration, contusion, blunt force trauma, ischemia, surgery, transplant, sunburn, chemical burn, high temperature burn, low temperature burn) or inflammatory symptoms associated with an inflammation caused by physical injury (such as, for example, abrasion, puncture, laceration, contusion, blunt force trauma, ischemia, surgery, transplant, sunburn, chemical burn, high temperature burn, low temperature burn) in a subject with an inflammation caused by physical injury (such as, for example, abrasion, puncture, laceration, contusion, blunt force trauma, ischemia, surgery, transplant, sunburn, chemical burn, high temperature burn, low temperature burn) comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy2l, antisense lnRNA selected from the group consisting of ANKRD5-1, ZNF236-1, and ACP1, and/or antisense RNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

In one aspect, it is understood and herein contemplated that one way to treat a wound is through administration of the secretome (including, but not limited to liposomes, lipid nanoparticles, MSC growth factor, MSC exosome, MSC extracts and/or extracellular vesicle comprising compositions) and RNA compositions subcutaneously, intramuscularly, intravenously, topically (such as, for example, through the use of salves, creams, and/or ointments), but also by impregnating stents, sponges, matrixes, scaffolds, bandages, dressing, sutures, grafts, surgical drapes, surgical adhesive, and/or staples with the MSC secretome compositions. Thus, in one aspect, disclosed herein are medicated stents, scaffolds, sponges, matrixes, adhesive bandages, wound dressings, grafts, surgical drapes, sutures, salves, creams, or wound adhesives comprising a therapeutically effective amount of the MSC secretome composition. The MSC secretome compositions (including, but not limited to MSC growth factor, MSC exosome, MSC extracts and/or extracellular vesicle comprising compositions), as noted above, can be administered topically and applied to the face, the neck, the hands, or any other desired part of the body. When applied to an adhesive bandage, wound dressing, grafts, surgical drape, suture, scaffold, matrix, sponge, or stent, the MSC secretome composition can be an applied as a powder.

### G. METHODS OF TREATING MICROBIAL DISEASE

In response to infection with a microbe such as, for example, a virus, bacterium, fungus, or parasite, the host immune system attempts to eliminate the infecting microbe by employing arms of the innate and adaptive immune systems including the production of cytokines, antibodies, and effector mechanisms of granulocyte, monocyte, macrophage, dendritic cell, innate lymphoid cells, NK cells, NK T cells, T cells, B cells, and plasma cells. In any microbial inflammation, inflammatory signaling cascades, which are initiated by cell responses to microbial virulence factors and endogenous cytokines, culminate in nuclear translocation of Stress-Responsive Transcription Factor (SRTFs) that upregulate inflammatory gene networks. Unchecked, this genomic reprogramming (genomic storm) leads to endothelial dysfunction, multi-organ failure and ultimately fatal shock, known as septic shock, that represents the ultimate end stage of microbial inflammation, one of the 10 leading causes of death in developed and developing countries.

“Microbial inflammation” refers to a condition associated with its cardinal signs such as redness, swelling, increase in temperature, pain, and impairment of organ function such as disordered respiration as a result of the epithelial injury with adjacent microvascular endothelial injury in the lungs (and other organs) due to a microbial infection such as a virus, bacteria, fungi, or parasite. That is, “Microbial inflammation” is a mechanism of disease caused by infection (“microbial insult”). Microbial inflammation evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the microbial inflammation and its end stage, sepsis can result from any microbial insult elicited by known (or unknown) virulence factors and microbial antigens.

The innate and adaptive immune response to infecting pathogen (disease-causing microorganism) can include the burst in production of cytokines, chemokines, and proteolytic enzymes by granulocytes, monocytes, macrophages, dendritic cells, mast cells, innate lymphoid cells, T cells, B cells, NK cells, and NK T cells. Microbial inflammation can be localized to a specific organ- or can be systemic. Microbial inflammation can proceed in stages from acute to subacute and chronic with attendant tissue destruction and subsequent fibrosis. Left unchecked, the acute microbial inflammation can lead to sepsis and septic shock, the end stage of microbial inflammation.

“Pathogen” is an agent that causes infection or disease, especially a virus, bacterium, fungus, protozoa, or parasite.

It is understood that the pathogen can be a virus. Thus in one embodiment the pathogen can be selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-0C43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods wherein the pathogen is a bacterium. The pathogen can be selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species.

Also disclosed are methods wherein the pathogen is a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium marneffi, and Alternaria alternata.

Also disclosed are methods wherein the pathogen is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

It is understood and herein contemplated that the microbial inflammation being treated can be in any tissue, organ, or system in the subject where a microbial infection can take place, including, but not limited to the blood, brain, sinuses, upper respiratory tract, or lungs heart, bone marrow, spleen, liver, kidneys, genito-urinary tract, bladder, aural cavities, stomach, intestines, skin, eyes, teeth, or gingiva, and musculoskeletal system. Thus, in one aspect, disclosed herein are methods of treating, inhibiting, decreasing, reducing, ameliorating and/or preventing microbial inflammation at its different stages (such as, for example, acute inflammation, subacute inflammation, chronic inflammation, organ-specific inflammation, systemic inflammation, and/or sepsis and septic shock as the end stage of microbial inflammation) in a subject comprising administering to the subject a therapeutically effective amount of any of the compositions disclosed herein (i.e., compositions comprising one or more secretomes and two or more RNAs). Accordingly, in one aspect disclosed herein are methods of treating, inhibiting, decreasing, reducing, ameliorating and/or preventing inflammatory injury resulting from a microbial infection in a subject comprising administering to the subject a composition comprising one or more secretomes and two or more RNAs comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy2l, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

Also disclosed herein are methods of treating, inhibiting, decreasing, reducing, ameliorating, and/or preventing inflammatory injury resulting from a microbial infection in a subject comprising administering to the subject a therapeutically effective amount of a composition comprising one or more secretomes and two or more RNA molecules, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p.

Also disclosed herein are methods of treating, inhibiting, decreasing, reducing, ameliorating and/or preventing inflammatory injury resulting from a microbial infection or inflammatory symptoms associated with an inflammatory injury resulting from a microbial infection in a subject with an inflammatory injury resulting from a microbial infection comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also disclosed herein are methods of treating, inhibiting, decreasing, reducing, ameliorating and/or preventing inflammatory injury resulting from a microbial infection or inflammatory symptoms associated with an inflammatory injury resulting from a microbial infection in a subject with an inflammatory injury resulting from a microbial infection comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one y-RNA selected from the group consisting of RF00019.441, RF00019.451, RF00019.621, RF00019.646, and RF00019.413. Also disclosed herein are methods of treating, inhibiting, decreasing, reducing, ameliorating and/or preventing inflammatory injury resulting from a microbial infection or inflammatory symptoms associated with an inflammatory injury resulting from a microbial infection in a subject with an inflammatory injury resulting from a microbial infection comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one lncRNA selected from the group consisting of LOC100129335-5, ZNF238, CADM2-7, SALL1-6, and VWA5A. Also disclosed herein are methods of treating, inhibiting, decreasing, reducing, ameliorating and/or preventing inflammatory injury resulting from a microbial infection or inflammatory symptoms associated with an inflammatory injury resulting from a microbial infection in a subject with an inflammatory injury resulting from a microbial infection comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one pi-RNA selected from the group consisting of piR-33044, piR-33043, and piR-31703. In one aspect, the RNA can be antisense RNA. Thus, also disclosed herein are methods of treating, inhibiting, decreasing, reducing, ameliorating and/or preventing inflammatory injury resulting from a microbial infection or inflammatory symptoms associated with an inflammatory injury resulting from a microbial infection in a subject with an inflammatory injury resulting from a microbial infection comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy2l, antisense lnRNA selected from the group consisting of ANKRD5-1, ZNF236-1, and ACP1, and/or antisense RNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy2l, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

In one aspect, the microbial inflammation is in the blood, brain, sinuses, upper respiratory tract, or lungs, heart, bone marrow, spleen, liver, kidneys, genito-urinary tract, bladder, aural cavities, stomach, intestines, skin, eyes, teeth, or gingiva and musculoskeletal system.

It is understood and herein contemplated that despite the ability of the disclosed compositions to inhibit microbial inflammation, there can be instances where the addition (either in the composition itself or as a separate administration) of an anti-microbial is desired for the treatment of the actual infection. Accordingly, disclosed herein are methods of treating, inhibiting, reducing, or preventing microbial inflammation in a subject, wherein the method further comprises administering to the subject an anti-microbial agent. Anti-microbial agents can comprise any antibiotics, antibodies, small molecules, and functional nucleic acids (siRNA, RNAi, anti-sense oligonucleotides), that directly attack the infecting microbe or alter host conditions rendering the host system inhospitable to the microbe. Such agents include, but are not limited to Abacavir, Acyclovir, Adefovir, Amantadine, Amprenavir, Ampligen, Arbidol, Atazanavir, Atripla, Balavir, Beta-D-N4-hydroxycitidine (NHC, EIDD-1931), Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Hydroxy-chloroquine, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Nitazoxanide, Norvir, Oseltamivir, Peginterferon alfa-2a, Penciclovir, Peramivir, Pleconaril, Podophyllotoxin, Raltegravir, Remdecivir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, Clofazimine; Dapsone; Capreomycin; Cycloserine; Ethambutol (Bs); Ethionamide; Isoniazid; Pyrazinamide; Rifampicin; Rifabutin; Rifapentine; Streptomycin; Arsphenamine; Chloramphenicol (Bs); Fosfomycin; Fusidic acid; Metronidazole; Mupirocin; Platensimycin; Quinupristin/Dalfopristin; Thiamphenicol; Tigecycline (Bs); Tinidazole; Trimethoprim (Bs); aminoglycosides such as, for example, Amikacin, Gentamicin, Kanamycin, Meropenem, Neomycin, Netilmicin, Tobramycin, Paromomycin, Streptomycin, Spectinomycin, Nitazoxanide, Melarsoprol Eflornithine, Metronidazole, Tinidazole, Miltefosine, Mebendazole, Pyrantel pamoate, Thiabendazole, Diethylcarbamazine, Ivermectin, Niclosamide, Praziquantel, Albendazole, Praziquantel, Rifampin, Amphotericin B, Fumagillin, Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Balsam of Peru, Orotomide, Miltefosine; ansamycins, such as, for example, geldanamycin, rifaximin, herbimycin; Carbapenems, such as, for example, Ertapenem, Doripenem, Imipenem/Cilastatin, and Meropenem; Cephalosporins, such as, for example, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole; Glycopeptides, such as, for example Teicoplanin, Vancomycin, Telavancin, Dalbavancin, and Oritavancin; Lincosamides (Bs), such as, for example, Clindamycin and Lincomycin; Lipopeptides, such as, for example, Daptomycin; Macrolides (Bs), such as, for example, Azithromycin, Clarithromycin, Erythromycin, Roxithromycin, Telithromycin, and Spiramycin; Monobactams, such as, for example, Aztreonam; Nitrofurans, such as, for example, Furazolidone and Nitrofurantoin (Bs); Oxazolidinones (Bs), such as, for example, Linezolid, Posizolid, Radezolid, and Torezolid; Penicillins, such as, for example, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Polypeptides, such as, for example, Bacitracin, Colistin, and Polymyxin B; Quinolones/Fluoroquinolones, such as, for example, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; Sulfonamides (Bs), such as, for example, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), and Sulfonamidochrysoidine (archaic); Tetracyclines (Bs), such as, for example, Demeclocycline, Doxycycline, Metacycline, Minocycline, Oxytetracycline, and Tetracycline; monoclonal antibodies such as, for example, Actoxumab, Atidortoxumab, Berlimatoxumab; Bezlotoxumab, Cosfroviximab, Edobacomab, Felvizumab, Firivumab, Foravirumab, Larcaviximab, Motavizumab, Navivumab, Panobacumab, Palivizumab, Porgaviximab, CR6261, Rafivirumab, Pagibaximab, Obiltoxaximab, Ibalizumab, Regavirumab, Rmab, Sevirumab, Rivabazumab pegol, Tefibazumab, Suvratoxumab, and Tuvirumab; and checkpoint inhibitors; Pembrolizumab, Nivolumab, Atezolizumab, Avelumab, Durvalumab, pidilizumab, AMP-224, AMP-514, PDR001, cemiplimab, and Ipilimumab.

### H. METHODS OF TREATING CANCER

The disclosed secretome and RNA compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers. Accordingly, in one aspect, disclosed herein are methods of treating, inhibiting, decreasing, reducing, ameliorating and/or preventing inflammatory injury resulting from a cancer and/or metastasis in a subject comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules comprising hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, hsa-miR-99b-5p, HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, NANS, LOC100129335-5, ZNF238, CADM2-7, SALL1-6, VWA5A, RF00019.441, RF00019.451, RF00019.621, RF00019.646, RF00019.413, piR-33044, piR-33043, piR-31703, RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and/or U7:RF00066.146, UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy21, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

For example, disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-197-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p. Preferably, disclosed herein are compositions, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-17-5p, miR-30a-5p, and miR-30d-5p. Also disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCMI, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS. Also disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one y-RNA selected from the group consisting of RF00019.441, RF00019.451, RF00019.621, RF00019.646, and RF00019.413. Also disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one lncRNA selected from the group consisting of LOC100129335-5, ZNF238, CADM2-7, SALL1-6, and VWA5A. Also disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one pi-RNA selected from the group consisting of piR-33044, piR-33043, and piR-31703. In one aspect, the RNA can be antisense RNA. Thus, also disclosed herein are methods of treating, inhibiting, decreasing, reducing, ameliorating and/or preventing a cancer and/or metastasis or inflammatory symptoms associated with a cancer in a subject with a cancer comprising administering to the subject a therapeutically effective amount of a composition comprising one or more secretomes (including, but not limited to liposomes, lipid nanoparticles, MSC growth factor, MSC exosome, MSC extracts and/or extracellular vesicle comprising compositions) and two or more RNA molecules, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy2l, antisense lnRNA selected from the group consisting of ANKRD5-1, ZNF236-1, and ACP1, and/or antisense RNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, GAGE2D:copy2l, ANKRD5-1, ZNF236-1, ACP1, EPHA1-AS1, FLJ45974, ANKRD30BP2, LINC00340, GRIK1-AS1, LOC440300, CSPG4P8, ADARB2-AS1, MLLT4-AS1, and/or HNRNPU-AS1.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### I. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In this example, the effect of naturally produced microRNAs packaged in bone marrow mesenchymal stem cell derived extracellular vesicles (BM-MSC EVs) was compared to the effect of artificially synthesized microRNAs of sequences shown to inhibit the formation of inflammasomes and to inhibit secretion of the pro-inflammatory cytokine, interleukin 1-beta (IL-1β).

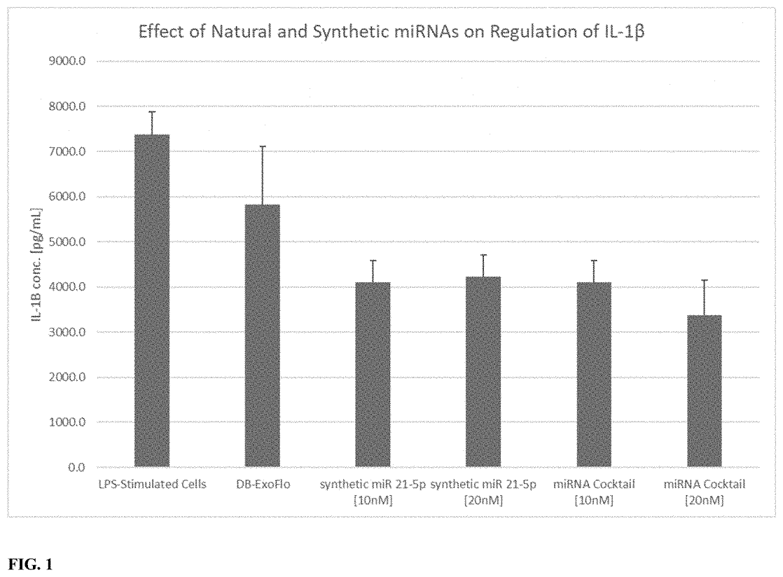

THP-1 cells were differentiated into macrophage phenotype cells and were exposed to LPS to induce IL-1β production by the cells. Cells were then exposed to either naturally generated RNA packaged in BM-MSC EVs or alternatively were transfected with either a cocktail of synthetic RNA sequences associated with the negative regulation of IL-1β. The miRNA cocktail included human miRNA sequences: miR-222-3p, miR-24-3p, miR-27b-3p, miR-221-3p, miR-21-5p. In one test condition, the microRNA hmiR21-5p was transfected alone since it is considered to be a primary miRNA regulatory of IL-1β. Twenty-four hours after addition of the LPS and test articles to the cells, conditioned media was collected, and IL-1β protein concentration was measured using a commercially available ELISA assay kit. The results of the study are shown in FIG. 1. The percent inhibition of IL-1β in test samples relative to LPS-stimulated cells alone is summarized in Table 1.

TABLE 1

| | % Inhibition from |
|---|---|
| Test Condition | Positive Control |
| DB-ExoFlo | 20.9% |
| synthetic miR 21-5p [10 nM] | 44.3% |
| synthetic miR 21-5p [20 nM] | 42.5% |
| miRNA Cocktail [10 nM] | 44.3% |
| miRNA Cocktail [20 nM] | 54.1% |

Inflammation modulation of naturally synthesized miRNAs packaged in BM-MSC EVs and that of synthetic miRNA mimic sequences reported in the literature to regulate IL-1B. Data is expressed as percent inhibition from the LPS-Stimulated Cells alone Control.

### J. SEQUENCES

miR-92a-3p stem loop sequence
SEQ ID NO: 1
CUUUCUACACAGGUUGGGAUCGGUUGCAAUGCUGUGUUUCUGUAUGGUAU
UGCACUUGUCCCGGCCUGUUGAGUUUGG
let-7i-5p stem loop sequence
SEQ ID NO: 2
CUGGCUGAGGUAGUAGUUUGUGCUGUUGGUCGGGUUGUGACAUUGCCCGC
UGUGGAGAUAACUGCGCAAGCUACUGCCUUGCUA
miR-16-5p stem loop sequence
SEQ ID NO: 3
GUCAGCAGUGCCUUAGCAGCACGUAAAUAUUGGCGUUAAGAUUCUAAAAU
UAUCUCCAGUAUUAACUGUGCUGCUGAAGUAAGGUUGAC
miR-93-5p stem loop sequence
SEQ ID NO: 4
CUGGGGGCUCCAAAGUGCUGUUCGUGCAGGUAGUGUGAUUACCCAACCUA
CUGCUGAGCUAGCACUUCCCGAGCCCCCGG
miR-21-5p stem loop sequence
SEQ ID NO: 5
UGUCGGGUAGCUUAUCAGACUGAUGUUGACUGUUGAAUCUCAUGGCAACA
CCAGUCGAUGGGCUGUCUGACA
miR-29c-3p stem loop sequence
SEQ ID NO: 6
AUCUCUUACACAGGCUGACCGAUUUCUCCUGGUGUUCAGAGUCUGUUUUU
GUCUAGCACCAUUUGAAAUCGGUUAUGAUGUAGGGGGA
miR-34a-5p stem loop sequence
SEQ ID NO: 7
GGCCAGCUGUGAGUGUUUCUUUGGCAGUGUCUUAGCUGGUUGUUGUGAGC
AAUAGUAAGGAAGCAAUCAGCAAGUAUACUGCCCUAGAAGUGCUGCACGU
UGUGGGGCCC
mir-203a-3p stem loop sequence
SEQ ID NO: 8
GUGUUGGGGACUCGCGCGCUGGGUCCAGUGGUUCUUAACAGUUCAACAGU
UCUGUAGCGCAAUUGUGAAAUGUUUAGGACCACUAGACCCGGCGGGCGCG
GCGACAGCGA
miR-27b-3p stem loop sequence
SEQ ID NO: 9
ACCUCUCUAACAAGGUGCAGAGCUUAGCUGAUUGGUGAACAGUGAUUGGU
UUCCGCUUUGUUCACAGUGGCUAAGUUCUGCACCUGAAGAGAAGGU
miR-140-3p stem loop sequence
SEQ ID NO: 10
UGUGUCUCUCUCUGUGUCCUGCCAGUGGUUUUACCCUAUGGUAGGUUACG
UCAUGCUGUUCUACCACAGGGUAGAACCACGGACAGGAUACCGGGGCACC
miR-24-3p stem loop sequence
SEQ ID NO: 11
CUCCGGUGCCUACUGAGCUGAUAUCAGUUCUCAUUUUACACACUGGCUCA
GUUCAGCAGGAACAGGAG
miR-222-3p stem loop sequence
SEQ ID NO: 12
GCUGCUGGAAGGUGUAGGUACCCUCAAUGGCUCAGUAGCCAGUGUAGAUC
CUGUCUUUCGUAAUCAGCAGCUACAUCUGGCUACUGGGUCUCUGAUGGCA
UCUUCUAGCU
miR-25-3p stem loop sequence
SEQ ID NO: 13
GGCCAGUGUUGAGAGGCGGAGACUUGGGCAAUUGCUGGACGCUGCCCUGG
GCAUUGCACUUGUCUCGGUCUGACAGUGCCGGCC
miR-10b-5p stem loop sequence
SEQ ID NO: 14
CCAGAGGUUGUAACGUUGUCUAUAUAUACCCUGUAGAACCGAAUUUGUGU
GGUAUCCGUAUAGUCACAGAUUCGAUUCUAGGGGAAUAUAUGGUCGAUGC
AAAAACUUCA
miR-221-3p stem loop sequence
SEQ ID NO: 15
UGAACAUCCAGGUCUGGGGCAUGAACCUGGCAUACAAUGUAGAUUUCUGU
GUUCGUUAGGCAACAGCUACAUUGUCUGCUGGGUUUCAGGCUACCUGGAA
ACAUGUUCUC
miR-17-5p stem loop sequence
SEQ ID NO: 16
GUCAGAAUAAUGUCAAAGUGCUUACAGUGCAGGUAGUGAUAUGUGCAUCU
ACUGCAGUGAAGGCACUUGUAGCAUUAUGGUGAC
miR-30a-5p stem loop sequence
SEQ ID NO: 17
GCGACUGUAAACAUCCUCGACUGGAAGCUGUGAAGCCACAGAUGGGCUUU
CAGUCGGAUGUUUGCAGCUGC
miR-30d-5p stem loop sequence
SEQ ID NO: 18
GUUGUUGUAAACAUCCCCGACUGGAAGCUGUAAGACACAGCUAAGCUUUC
AGUCAGAUGUUUGCUGCUAC
miR-92a-3p mature sequence
SEQ ID NO: 19
UAUUGCACUUGUCCCGGCCUGU
let-7i-5p mature sequence
SEQ ID NO: 20
UGAGGUAGUAGUUUGUGCUGUU
miR-16-5p mature sequence
SEQ ID NO: 21
UAGCAGCACGUAAAUAUUGGCG
miR-93-5p mature sequence
SEQ ID NO: 22
CAAAGUGCUGUUCGUGCAGGUAG
miR-21-5p mature sequence
SEQ ID NO: 23
UAGCUUAUCAGACUGAUGUUGA
miR-214-3p mature sequence
SEQ ID NO: 24
ACAGCAGGCACAGACAGGCAGU
miR-29c-3p mature sequence
SEQ ID NO: 25
UAGCACCAUUUGAAAUCGGUUA
miR-34a-5p mature sequence
SEQ ID NO: 26
UGGCAGUGUCUUAGCUGGUUGU
mir-203a-3p mature sequence
SEQ ID NO: 27
GUGAAAUGUUUAGGACCACUAG
miR-27b-3p mature sequence
SEQ ID NO: 28
UUCACAGUGGCUAAGUUCUGC
miR-140-3p mature sequence
SEQ ID NO: 29
UACCACAGGGUAGAACCACGG
miR-24-3p mature sequence
SEQ ID NO: 30
UGGCUCAGUUCAGCAGGAACAG
miR-222-3p mature sequence
SEQ ID NO: 31
AGCUACAUCUGGCUACUGGGU
miR-25-3p mature sequence
SEQ ID NO: 32
CAUUGCACUUGUCUCGGUCUGA
miR-106-5p mature sequence
SEQ ID NO: 33
UACCCUGUAGAACCGAAUUUGUG
miR-221-3p mature sequence
SEQ ID NO: 34
AGCUACAUUGUCUGCUGGGUUUC
miR-17-5p mature sequence
SEQ ID NO: 35
CAAAGUGCUUACAGUGCAGGUAG
miR-30a-5p mature sequence
SEQ ID NO: 36
UGUAAACAUCCUCGACUGGAAG
miR-30d-5p mature sequence
SEQ ID NO: 37
UGUAAACAUCCCCGACUGGAAG

What is claimed is:

1. A composition comprising one or more secretomes and two or more ribonucleic nucleic acids (RNAs).

2. The composition of claim 1, wherein the secretome comprises liposomes or lipid nanoparticles.

3. The composition of claim 1, wherein the secretome comprises a mesenchymal stem cell (MSC) derived exosome or extracellular vesicle.

4. The composition of claim 1, wherein the two or more RNA comprise microRNA (miRNA), messenger RNA (mRNA), long non-coding RNA (lncRNA), y-RNA, and/or pi-RNA.

5. The composition of claim 4, wherein the two or more RNA comprises at least one RNA selected from the group consisting of RNY4, LOC441666, SNHG1, RNU11, LOC80154, LOC727751, LOC388152, GOLGA2P2Y, GOLGA2P3Y, SNHG12, RNU12, ASMTL-AS1, LOC284454 U2:RF00004.39, U2:RF00004.22, U2:RF00004.35, U2:RF00004.71, U2:RF00004.66 U2:RF00004.91, U2:RF00004.89, U2:RF00004.67, U1:RF00003.5, U1:RF00003.109, U1:RF00003.110, U1:RF00003.8, U1:RF00003.3, U1:RF00003.6, U1:RF00003.38, U1:RF00003.20, U1:RF00003.24, U1:RF00003.73, U1:RF00003.11, U1:RF00003.27, U1:RF00003.40, U1:RF00003.107, U1:RF00003.62, U1:RF00003.45, U1:RF00003.116, U1:RF00003.126, U1:RF00003.81, U1:RF00003.100, U1:RF00003.57, U1:RF00003.95, U1:RF00003.31, U1:RF00003.61, U1:RF00003.25, U1:RF00003.90, U1:RF00003.46, U1:RF00003.123, U2:RF00004.42, U2:RF00004.61, U2:RF00004.51, U1:RF00003.122, U1:RF00003.22, U1:RF00003.9, snoMBII-202:RF00324.2, let-7:RF00027.7, U2:RF00004.75, U1:RF00003.69, U2:RF00004.88, U12:RF00007.1, U7:RF00066.141, U1:RF00003.85, U7:RF00066.49, U2:RF00004.77, U1:RF00003.113, U1:RF00003.112, U1:RF00003.70, U1:RF00003.83, U7:RF00066.89, U7:RF00066.124, U7:RF00066.75, U7:RF00066.77, U7:RF00066.118, U7:RF00066.103, U7:RF00066.79, U7:RF00066.200, and U7:RF00066.146.

6. The composition of claim 4, wherein the two or more RNA comprises at least one antisense RNA selected from the group consisting of EPHA1-AS1, F1145974, ANKRD30BP2, LINC00340, GRIK1-AS1, LOC440300, CSPG4P8, ADARB2-AS1, MLLT4-AS1, and HNRNPU-AS1.

7. The composition of claim 4, wherein the two or more RNA comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-5′70-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

8. The composition of claim 6, wherein the two or more RNA comprises at least one miRNA selected from the group consisting of miR-92a-3p, let-7i-5p, miR-16-5p, miR-93-5p, miR-21-5p, miR-214-3p, miR-29c-3p, miR-34a-5p, mir-203a-3p, miR-27b-3p, miR-140-3p, miR-24-3p, miR-222-3p, miR-25-3p, miR-10b-5p, miR-221-3p, miR-1′7-5p, miR-30a-5p, and miR-30d-5p.

9. The composition of claim 4, wherein the two or more RNA comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCM7, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS.

10. The composition of claim 4, wherein the two or more RNA comprises at least one antisense mRNA selected from the group consisting of UBAP11, SMC51, TMX11, LTA4H1, KIF20B1, OPCML1, RNLS1, CDKL21, C10orf531, ACOX31, GPC51, GATA41, EPHA61, CNTNAP51, ZRANB31, KIAA17151, TOX1, ALPK11, TACR11, ZNF6521, FAM154A1, NAA351, HS3ST41, SLC44A51, CLCN51, ST6GALNAC51, ABCA41, COL5A11, AKIP11, UGT81, DRP21, AMOTL11, FGFR1OP21, WBSCR271, NBPF141, TMEM132E1, GAGE131, GAGE2D1, and GAGE2D:copy21.

11. The composition of claim 4, wherein the two or more RNA comprises at least one lncRNA selected from the group consisting of LOC100129335-5, ZNF238, CADM2-7, SALL1-6, and VWA5A.

12. The composition of claim 4, wherein the two or more RNA comprises at least one antisense lncRNA selected from the group consisting of ANKRD5-1, ZNF236-1, and ACP1.

13. The composition of claim 4, wherein the two or more RNA comprises at least one y-RNA selected from the group consisting of RF00019.441, RF00019.451, RF00019.621, RF00019.646, and RF00019.413.

14. The composition of claim 4, wherein the two or more RNA comprises at least one pi-RNA selected from the group consisting of piR-33044, piR-33043, and piR-31703.

15. The composition of claim 1, wherein the RNA comprises a modification to reduce degradation.

16. The composition of claim 15, wherein the modification comprises the addition of 2′-(O)-Methyl, 2′-Fluoro, or 2′-Hydro groups.

17. A method of modulating an immune response to an inflammatory injury in a subject comprising administering to the subject a composition comprising one or more secretomes and two or more ribonucleic nucleic acids (RNAs).

18. The method of modulating an immune response of claim 17, wherein the modulation increases inflammation and wherein the two or more RNA of the composition comprises at least one mRNA selected from the group consisting of HBII-202, U26, U29, HBII-420, U30, U18A, U81, GSKIP, IFT88, PCDH11X, PCDH11Y, KIAA0226L, PDE11A, PHACTR1, TEX14, DCUN1D2, SIM1, FILIP1, FAM107B, CHADL, ZNF365, MNAT1, CYSTM1, GAS7, HOXB7, ASTN2, LGR5, ZFHX3, MCM7, GRIK1, MAP2, LHFPL3, PTER, STAMBP, CCDC135, PAX9, RPL13, ABCA13, DAAM1, OPN1LW, OPN1MW2, CNOT6L, MTRNR2L10, SMC4, RPL4, PSAP, ASIC4, AHCYL1, RD3L, SIGLEC1, SYCE2, and NANS.

19. The method of modulating an immune response of claim 17, wherein the modulation decreases inflammation and wherein the two or more RNA of the composition comprises at least one miRNA selected from the group consisting of hsa-let-7a-5p, hsa-let-7b-5p, hsa-let-7c-5p, hsa-let-7d-3p, hsa-let-7e-5p, hsa-let-7g-5p, hsa-let-7i, hsa-let-7i-5p, hsa-miR-100-5p, hsa-miR-103a-3p, hsa-miR-106a-5p, hsa-miR-106b-5p, hsa-mir-10b, hsa-miR-10b-5p, hsa-mir-1246, hsa-miR-1246, hsa-miR-125a-5p, hsa-miR-125b-5p, hsa-miR-130a-3p, hsa-mir-130b, hsa-miR-130b-3p, hsa-miR-132-3p, hsa-miR-136-5p, hsa-miR-138-5p, hsa-miR-139-5p, hsa-mir-140, hsa-miR-140-3p, hsa-miR-145-5p, hsa-mir-146a, hsa-miR-146a-5p, hsa-miR-148a-3p, hsa-miR-152-3p, hsa-miR-15a-5p, hsa-miR-15b-5p, hsa-mir-16-1, hsa-mir-16-2, hsa-miR-16-5p, hsa-miR-17-5p, hsa-miR-181a-5p, hsa-miR-191-5p, hsa-miR-193a-5p, hsa-miR-193b-3p, hsa-miR-19′7-3p, hsa-miR-199a-3p, hsa-miR-199a-5p, hsa-miR-199b-5p, hsa-miR-19a-3p, hsa-miR-19b-3p, hsa-miR-20a-5p, hsa-miR-203a, hsa-miR-203a-3p, hsa-miR-214-3p, hsa-mir-21, hsa-miR-21-3p, hsa-miR-21-5p, hsa-mir-221, hsa-miR-221-3p, hsa-mir-222, hsa-miR-222-3p, hsa-miR-22-3p, hsa-miR-23a-3p, hsa-miR-23b-3p, hsa-mir-24-1, hsa-mir-24-2, hsa-miR-24-3p, hsa-mir-25, hsa-miR-25-3p, hsa-miR-26a-5p, hsa-miR-27a-3p, hsa-mir-27b, hsa-miR-27b-3p, hsa-miR-29a-3p, hsa-miR-29c-3p, hsa-miR-30a-5p, hsa-miR-30a-5p, hsa-miR-30b-5p, hsa-miR-30c-5p, hsa-mir-30d, hsa-miR-30d-5p, hsa-mir-30e, hsa-miR-30e-5p, hsa-miR-31-3p, hsa-miR-31-5p, hsa-miR-320a, hsa-miR-342-3p, hsa-miR-345-5p, hsa-miR-34a-5p, hsa-miR-361-5p, hsa-miR-376a-3p, hsa-miR-376c-3p, hsa-miR-423-3p, hsa-miR-423-5p, hsa-miR-424-5p, hsa-miR-484, hsa-mir-486-1, hsa-mir-486-2, hsa-miR-486-5p, hsa-miR-570-3p, hsa-miR-574-3p, hsa-miR-663a, hsa-miR-874-3p, hsa-mir-92a-1, hsa-mir-92a-2, hsa-miR-92a-3p, hsa-miR-92b-3p, hsa-mir-93, hsa-miR-93-5p, hsa-miR-940, hsa-miR-99a-5p, and hsa-miR-99b-5p.

20. A method of treating an autoimmune disease, an autoinflammatory disease, or an inflammatory injury in a subject comprising administering to the subject a composition comprising one or more secretomes and two or more ribonucleic nucleic acids (RNAs), wherein the inflammatory injury results from a physical insult, a microbial infection, or a cancer.

21. (canceled)

22. (canceled)

23. (canceled)

24. (canceled)


SEQUENCE LISTING (37 sequences)

SEQ ID NO: 1 - RNA, 78 nt, unidentified
  source 1..78: mol_type = unassigned RNA
ctttctacacaggttgggatcggttgcaatgctgtgtttctgtatggtattgcacttgtcccggcctgttgagtttgg

SEQ ID NO: 2 - RNA, 84 nt, unidentified
  source 1..84: mol_type = unassigned RNA
ctggctgaggtagtagtttgtgctgttggtcgggttgtgacattgcccgctgtggagataactgcgcaagctactgccttgcta

SEQ ID NO: 3 - RNA, 89 nt, unidentified
  source 1..89: mol_type = unassigned RNA
gtcagcagtgccttagcagcacgtaaatattggcgttaagattctaaaattatctccagtattaactgtgctgctgaagtaaggttgac

SEQ ID NO: 4 - RNA, 80 nt, unidentified
  source 1..80: mol_type = unassigned RNA
ctgggggctccaaagtgctgttcgtgcaggtagtgtgattacccaacctactgctgagctagcacttcccgagcccccgg

SEQ ID NO: 5 - RNA, 72 nt, unidentified
  source 1..72: mol_type = unassigned RNA
tgtcgggtagcttatcagactgatgttgactgttgaatctcatggcaacaccagtcgatgggctgtctgaca

SEQ ID NO: 6 - RNA, 88 nt, unidentified
  source 1..88: mol_type = unassigned RNA
atctcttacacaggctgaccgatttctcctggtgttcagagtctgtttttgtctagcaccatttgaaatcggttatgatgtaggggga

SEQ ID NO: 7 - RNA, 110 nt, unidentified
  source 1..110: mol_type = unassigned RNA
ggccagctgtgagtgtttctttggcagtgtcttagctggttgttgtgagcaatagtaaggaagcaatcagcaagtatactgccctagaagtgctgcacgttgtggggccc

SEQ ID NO: 8 - RNA, 110 nt, unidentified
  source 1..110: mol_type = unassigned RNA
gtgttggggactcgcgcgctgggtccagtggttcttaacagttcaacagttctgtagcgcaattgtgaaatgtttaggaccactagacccggcgggcgcggcgacagcga

SEQ ID NO: 9 - RNA, 96 nt, unidentified
  source 1..96: mol_type = unassigned RNA
acctctctaacaaggtgcagagcttagctgattggtgaacagtgattggtttccgctttgttcacagtggctaagttctgcacctgaagagaaggt

SEQ ID NO: 10 - RNA, 100 nt, unidentified
  source 1..100: mol_type = unassigned RNA
tgtgtctctctctgtgtcctgccagtggttttaccctatggtaggttacgtcatgctgttctaccacagggtagaaccacggacaggataccggggcacc

SEQ ID NO: 11 - RNA, 68 nt, unidentified
  source 1..68: mol_type = unassigned RNA
ctccggtgcctactgagctgatatcagttctcattttacacactggctcagttcagcaggaacaggag

SEQ ID NO: 12 - RNA, 110 nt, unidentified
  source 1..110: mol_type = unassigned RNA
gctgctggaaggtgtaggtaccctcaatggctcagtagccagtgtagatcctgtctttcgtaatcagcagctacatctggctactgggtctctgatggcatcttctagct

SEQ ID NO: 13 - RNA, 84 nt, unidentified
  source 1..84: mol_type = unassigned RNA
ggccagtgttgagaggcggagacttgggcaattgctggacgctgccctgggcattgcacttgtctcggtctgacagtgccggcc

SEQ ID NO: 14 - RNA, 110 nt, unidentified
  source 1..110: mol_type = unassigned RNA
ccagaggttgtaacgttgtctatatataccctgtagaaccgaatttgtgtggtatccgtatagtcacagattcgattctaggggaatatatggtcgatgcaaaaacttca

SEQ ID NO: 15 - RNA, 110 nt, unidentified
  source 1..110: mol_type = unassigned RNA
tgaacatccaggtctggggcatgaacctggcatacaatgtagatttctgtgttcgttaggcaacagctacattgtctgctgggtttcaggctacctggaaacatgttctc

SEQ ID NO: 16 - RNA, 84 nt, unidentified
  source 1..84: mol_type = unassigned RNA
gtcagaataatgtcaaagtgcttacagtgcaggtagtgatatgtgcatctactgcagtgaaggcacttgtagcattatggtgac

SEQ ID NO: 17 - RNA, 71 nt, unidentified
  source 1..71: mol_type = unassigned RNA
gcgactgtaaacatcctcgactggaagctgtgaagccacagatgggctttcagtcggatgtttgcagctgc

SEQ ID NO: 18 - RNA, 70 nt, unidentified
  source 1..70: mol_type = unassigned RNA
gttgttgtaaacatccccgactggaagctgtaagacacagctaagctttcagtcagatgtttgctgctac

SEQ ID NO: 19 - RNA, 22 nt, unidentified
  source 1..22: mol_type = unassigned RNA
tattgcacttgtcccggcctgt

SEQ ID NO: 20 - RNA, 22 nt, unidentified
  source 1..22: mol_type = unassigned RNA
tgaggtagtagtttgtgctgtt

SEQ ID NO: 21 - RNA, 22 nt, unidentified
  source 1..22: mol_type = unassigned RNA
tagcagcacgtaaatattggcg

SEQ ID NO: 22 - RNA, 23 nt, unidentified
  source 1..23: mol_type = unassigned RNA
caaagtgctgttcgtgcaggtag

SEQ ID NO: 23 - RNA, 22 nt, unidentified
  source 1..22: mol_type = unassigned RNA
tagcttatcagactgatgttga

SEQ ID NO: 24 - RNA, 22 nt, unidentified
  source 1..22: mol_type = unassigned RNA
acagcaggcacagacaggcagt

SEQ ID NO: 25 - RNA, 22 nt, unidentified
  source 1..22: mol_type = unassigned RNA
tagcaccatttgaaatcggtta

SEQ ID NO: 26 - RNA, 22 nt, unidentified
  source 1..22: mol_type = unassigned RNA
tggcagtgtcttagctggttgt

SEQ ID NO: 27 - RNA, 22 nt, unidentified
  source 1..22: mol_type = unassigned RNA
gtgaaatgtttaggaccactag

SEQ ID NO: 28 - RNA, 21 nt, unidentified
  source 1..21: mol_type = unassigned RNA
ttcacagtggctaagttctgc

SEQ ID NO: 29 - RNA, 21 nt, unidentified
  source 1..21: mol_type = unassigned RNA
taccacagggtagaaccacgg

SEQ ID NO: 30 - RNA, 22 nt, unidentified
  source 1..22: mol_type = unassigned RNA
tggctcagttcagcaggaacag

SEQ ID NO: 31 - RNA, 21 nt, unidentified
  source 1..21: mol_type = unassigned RNA
agctacatctggctactgggt

SEQ ID NO: 32 - RNA, 22 nt, unidentified
  source 1..22: mol_type = unassigned RNA
cattgcacttgtctcggtctga

SEQ ID NO: 33 - RNA, 23 nt, unidentified
  source 1..23: mol_type = unassigned RNA
taccctgtagaaccgaatttgtg

SEQ ID NO: 34 - RNA, 23 nt, unidentified
  source 1..23: mol_type = unassigned RNA
agctacattgtctgctgggtttc

SEQ ID NO: 35 - RNA, 23 nt, unidentified
  source 1..23: mol_type = unassigned RNA
caaagtgcttacagtgcaggtag

SEQ ID NO: 36 - RNA, 22 nt, unidentified
  source 1..22: mol_type = unassigned RNA
tgtaaacatcctcgactggaag

SEQ ID NO: 37 - RNA, 22 nt, unidentified
  source 1..22: mol_type = unassigned RNA
tgtaaacatccccgactggaag